(19) United States
(12) Patent Application Publication
(10) Pub. No.: US 2006/0240571 A1
(43) Pub. Date: Oct. 26, 2006

(54) Biosensors and methods for detecting agents based upon time resolved luminescent resonance energy transfer

(76) Inventor: Joseph Edward Zahner, St. Louis, MO (US)

Correspondence Address: Joseph E. Zahner, 3646 Dover Place, St. Louis, MO, 63116, US

(21) Appl. No.: 11/408,529

(22) Filed: Apr. 20, 2006

Related U.S. Application Data
(60) Provisional application No. 60/673,059, filed on Apr. 20, 2005.

(51) Int. Cl. C12M 1/34; G01N 33/543

(52) U.S. Cl. 436/518; 435/287.2

(57) ABSTRACT

Disclosed are biosensors useful in the detection of potentially harmful or undesirable agents, particularly chemicals and microorganisms in food and water. The biosensors operate under the principle of time-resolved luminescence resonance energy transfer. In a preferred embodiment, the biosensor comprises at antibodies that recognized different but proximal epitopes on a particular agent. One antibody contains a luminescence donor that emits energy over time, such as a lanthanide series-based luminophor. Another antibody contains a luminescence acceptor that is excited by the emission spectrum of the donor and emits at a particular wavelength, such as for example the fluorophor Cy3. In the presence of the agent, the donor and acceptor are brought into close proximity, such that the energy transfer can occur. The donor is excited by a transient burst of light and the emitted wavelength is received by a photodiode, quantified and correlated to amount of agent in a sample.

This application claims priority to provisional patent application No. 60/673,059, which was filed on Apr. 20, 2005.

### BACKGROUND OF THE INVENTION

1. Field of the Invention

The invention is directed generally to a method and device for rapid detection and quantification of particular agents in a sample. The invention is directed specifically to the rapid detection and quantification of pathogens in food.

2. Description of the Related Art

### Food Safety

With increasing globalization and efficient food production and distribution, the assurance of the safety of our food supply is becoming an increasingly more important consideration. In 2003 in the United States alone, 76 million cases of food poisoning were reported, which represents a 6-fold increase since 1999. That same year, 325,000 hospitalizations and 5,000 deaths due to food pathogens were reported, which represent respectively a 6.4-fold increase and 3.8-fold increase since 1999. To help mitigate this rapidly growing public health problem and biosecurity issue, government agencies such as the U.S. FDA, USDA and Department of Homeland Security are directing significant resources toward the development of fast and portable food pathogen detectors.

The key food pathogens are listed in Table 1, along with U.S. morbidity and mortality statistics from 1999.

TABLE 1

| Pathogen | Illnesses | Hospitalizations | Deaths |
|---|---|---|---|
| Campylobacter | 2,000,000 | 10,500 | 99 |
| Salmonella | 1,300,000 | 16,000 | 556 |
| E. coli 0157:H7 | 62,500 | 1,800 | 52 |
| L. monocytogenes | 2,500 | 2,300 | 499 |
| viruses | 9,200,000 | 20,000 | 120 |
| Total | 12,658,000 | 50,600 | 1326 |

### Microbial Testing

Standard microbiological testing, which is used to determine the type and amount of a particular bacterial, viral, protest or fungal contamination, is both time consuming, expensive and technically demanding. This type of testing requires that a food sample be processed and then plated onto or inoculated into selective microbiological media. After 12 to 48 hours of growth, bacteria can be identified and correlated to a bacteria load in the original sample. The long delay between obtaining a sample and making a determination whether an unacceptable pathogen or bacteria load exists presents considerable problems, including loss of inventory, inventory storage problems, the cost of food recalls and accidental release of pathogens into the food chain.

Recent advances in molecular biology and sensor technology are enabling a new generation of food safety biosensors that are both highly sensitive and fast. Dupont, for example, are marketing a real-time PCR system for pathogen identification based upon unique DNA sequence probes. However, most of the systems for pathogen or toxin detection on the market today rely on the time consuming steps of enrichment and expansion of the number of pathogens prior to detection and identifiction. For a review on the development of rapid pathogen detection systems, see Benoit and Donahue, “Methods for rapid separation and concentration of bacteria in food that bypass time-consuming cultural enrichment,” J. Food Prot. Vol.66 (10), pp. 1935-1948, October 2003, which is incorporated herein by reference.

### Biosensors

Chemical and biological sensors may be divided into two general categories: specific and non-specific. Biosensors traditionally are specific, in that the chemistry of detection is based upon specific molecular interactions (e.g., the antibody interaction described above). Specific sensors may use receptors, antibodies, ligands or aptamers to capture or bind to a particular agent, upon which time a signal is transduced to signify the detection event. For example, a biosensor for the food pathogen Salmonella may use an antibody that specifically recognizes a cell surface molecule specific only to Salmonella and not to other food pathogens. Biosensors may be non-homogenous, which requires secondary processing steps to detect a signal, or homogenous, wherein the interaction of the agent-to-be-detected and the biosensor occurs in a simple and rapid step, which does not require extensive and time consuming use of reagents and washing steps to detect an interaction above background.

Biological sensors may utilize any one or more modes for transducing a molecular interaction event into a quantifiable signal. Those modes include mass detection, optical detection, piezoelectric detection, and other luminescence and electrical detection.

Mass detection is based upon an increase in mass at the sensor head upon binding of the specific agent. Surface plasmon resonance (SPR) utilizes a biospecific receptor bound to a chip, which changes its reflective properties when an agent has bound to the receptor. This assay is theoretically homogenous, meaning that several washing steps and the application of additional reagents is theoretically not required. For a more detailed description of surface plasmon resonance, see Karlsson, “SPR for molecular interaction analysis: a review of emerging application areas,” J Mol Recognit, May-June 2004; 17(3):151-61, which is incorporated herein by reference. Vibration-based sensors measure a change in vibration frequency of a biosensor chip, when an agent is bound to a receptor. This assay is also theoretically homogenous. In both of these mass detection motifs, the sensor surface may be coated with any one or more biomolecular recognition factors such as aptamers, antibodies, ligands and receptors.

Optical-based sensors measure a change in luminescence that occurs upon the binding of an agent to its biomolecular recognition factor on the biosensor surface. (1) Traditional fluorescent antibody sandwich and competition assays are well known and accepted methods for detecting agents. Generally, a fluorescent-labeled secondary antibody must be applied to the biosensor surface after the agent has been captured by an unlabelled primary antibody. This assay is not homogenous and requires a minimum of two hours to complete. For a review of antibody-based sensors, see Luppa et al., “Immunosensors—principles and applications to clinical chemistry,” Clin. Chim. Acta., 314 (1-2): 1-26, December 2001, which is incorporated herein by reference.

(2) Cell-based luminescence assays utilize living genetically engineered cells to detect an agent. For example a genetically engineered cell containing an agent-specific ion channel gated channel, which opens when the agent is bound to stimulate a Ca2+ response, can result in a detectable bioluminescence reaction. This method presents considerable logistical, technical expertise and equipment requirements. Theoretically, this is a homogenous sensor.

(3) Phosphorescence and chemiluminescence assays are generally non-specific and narrow in their applicability. However, genetically modified cells may be used as sensor devices. A common chemiluminescence biosensor uses luciferase, which emits light in the presence of ATP, which is indicative of a living agent being present. See Valat et al., “Use of ATP bioluminescence to determine the bacterial sensitivity threshold to a bacteriocin,” Luminescence. September-October 2003;18(5):254-8, which is incorporated herein by reference, for a review on Luciferase-based biosensors.

(4) Molecular beacons are synthetic nucleic acid aptamers containing luminescent or fluorescent moieties. Aptamers can be designed to specifically bind to a specific agent. In an unbound state, the aptamer is in a closed conformation, which shields the fluorescent moiety from absorbing or emitting any light. Upon binding an agent, the aptamer changes its conformation, thereby allowing the fluorescent moiety to fluoresce. This is a homogenous assay. For a review of molecular beacons, see Drake T J, and Tan W., “Molecular beacon DNA probes and their bioanalytical applications,” Appl Spectrosc. September 2004;58(9):269A-280A, which is incorporated herein by reference.

Piezoelectric sensors rely on changes in electrical current, voltage and/or resistance to quantify a biomolecular interaction. (1) In enzyme-based piezoelectric sensors, an oxidoreductase enzyme is used as a detectable marker for specific agent-receptor interaction. This assay is much like the antibody sandwich assay, where instead of a photon being emitted, an electron is released to produce a piezoelectric current. This is a non-homogenous, labor-intensive assay for most agents, with the notable exception of the glucose sensor, wherein glucose serves directly as the substrate for glucose oxidase in the biosensor.

(2) In ion channel-based piezoelectric sensors, biomolecular membranes comprising an agent-specific receptor-ion channel nano assembly are employed. Specially engineered membrane gates open or close to allow or disallow the passage of ions. This permits a measurable change in conductance. See D. Anrather et al., “Supported membrane nanodevices,” J Nanosci Nanotechnol. January-February 2004; 4(1-2):1-22, which is incorporated herein by reference.

(3) Various conducting polymers (e.g., polymethylene green) can be engineered to change in conductance upon the binding of an agent to a specific receptor on the polymer. A measurable change in conductance relative to a baseline can indicate an agent-receptor binding event. A variation on this theme is the Fluorescent Polymer QTL approach developed at Los Alamos and UCLA, wherein the polymers emit light instead of an electrical signal upon ligand-receptor interaction. See S. Kumaraswamy et al., “Fluorescent-conjugated polymer superquenching facilitates highly sensitive detection of proteases,” PNAS, May 18, 2004, vol. 101, no. 20, pp. 7511-7515, which is incorporated herein by reference.

### SUMMARY OF THE INVENTION

In one embodiment, the invention is directed to a biosensor comprising a first molecular recognition element labeled with a luminescence donor and a second molecular recognition element labeled with a luminescence acceptor. Preferably each molecular recognition element is an antibody, such that the first antibody and the second antibody bind to different but proximal epitopes on the agent to allow for the donor and acceptor to come within a Förster radius of each other to allow the transfer of luminescence energy from the donor to the acceptor. A preferred donor is a lanthanide chelate and a preferred acceptor is an organic dye, which can accept the energy from the lanthanide chelate and emit a light having a wavelength distinguishable from an emission wavelength of the lanthanide chelate. In one aspect, one or both molecular recognition entities are fixed to a substrate, such as for example a silicon chip, glass slide or the like.

In another embodiment, the invention is directed to a method of detecting an agent in a sample comprising the steps of applying a sample to a biosensor (such as the biosensor supra), exposing the biosensor to a pulse of light which excites the donor, quantitatively detecting light having an emission wavelength associated with the acceptor, and estimating the amount of agent present in the sample. Preferably, the sample is a food sample and the agent is a food pathogen.

### DESCRIPTION OF THE DRAWINGS

The following drawings form part of the present specification and are included to further demonstrate certain aspects of the present invention. The invention may be better understood by reference to one or more of these drawings in combination with the description of the invention presented herein:

FIG. 1 depicts a schematized biosensor. In this particular non-limiting embodiment, (a) the first molecular recognition element is an antibody conjugated to a luminescence energy donor (in this case a lanthanide chelate), (b) the second molecular recognition element is an antibody conjugated to a luminescence energy acceptor (in this case an organic dye). In the absence of the agent (left side of figure), which in this case is a bacteria antigen, the distance between the first molecular recognition element and the second molecular recognition element (r) is greater than the maximum distance between donor and recipient to allow for at least 10% efficiency of energy transfer between donor and recipient (X). In that case, no LRET signal is generated. In the presence of the agent (right side of figure), the first and second molecular recognition elements bind to their respective recognition sites on the agent in sufficient proximity to one another (i.e., r≦X) to allow for the LRET event to occur and a signal to be generated.

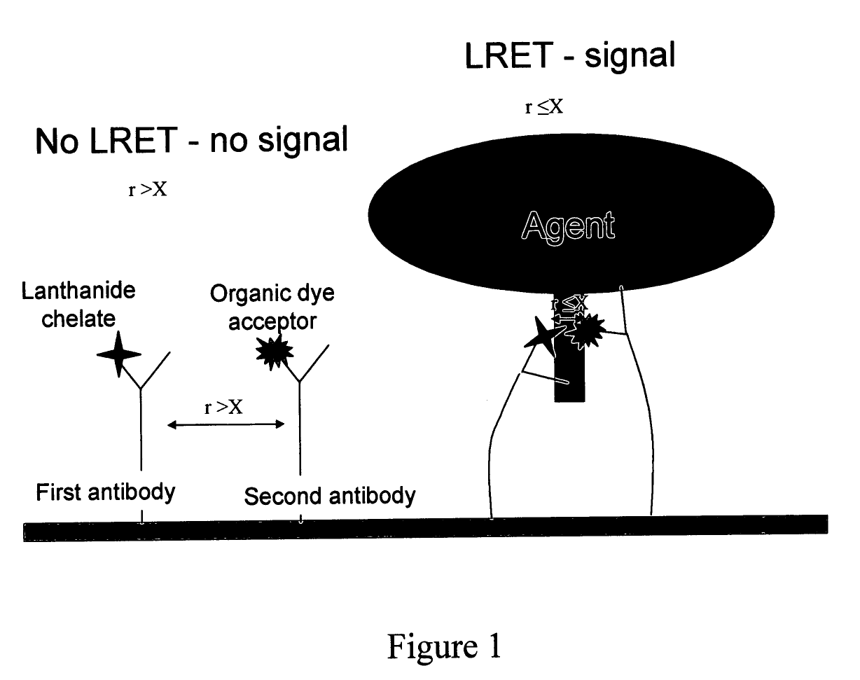

FIG. 2 depicts the result of two independent experiments conducted using a biosensor directed to the E. coli bacterium. Four (4) off-the-shelf monoclonal antibodies were pooled into two groups of two (2) antibodies, the first group labeled with a terbium chelate (donor) to form the first molecular recognition element (MRE) and the second group was labeled with the organic dye Cy3 to form the second MRE. Serial dilutions of Escherichia coli bacteria (E. coli) were made to which the first and second MREs were added. Within from five (5) to 15 minutes of mixing, each sample was subjected to 30 cycles per second of 5 ns pulses from a nitrogen laser (337 nm). Between light pulses (hence—time resolved) light of 541 nm (terbium emission) and 570 nm (Cy3 emission) were measured. The ratio of 570/541 was determined for each sample. A baseline of 1 was established as the 570/541 value for the E. coli—free control, and the relative ratio is presented on the Y-axis. The X-axis represents the total number of E. coli bacteria in each sample.

### DESCRIPTION OF THE INVENTION

The inventor has developed a device and method for detecting agents using time-resolved luminescence resonance energy transfer. Both the device and method comprise a biosensor component. In another embodiment, the device and method comprise a biosensor component and a hardware component to identify and quantify an agent. The biosensor component comprises two molecular recognition elements, each of which can bind to an agent at a particular spot on the agent. A luminescence energy donor moiety (“donor”) is linked to one of the molecular recognition elements (“first element”) and a luminescence energy recipient moiety (“recipient”) is linked to another molecular recognition element (“second element”). The hardware component comprises a light source capable of exciting the donor and a photodetector capable of detecting light emitted by the recipient excitation by the luminescence energy donor molecule. A molecular recognition element may be any chemical entity that binds to another chemical entity, such as for example but not limited to an antibody, an antigen, a ligand, a receptor, a product, a substrate, an enzyme, a polynucleotide, and any fragments thereof.

To operate the invention, a sample, which is suspected of containing an agent, is collected from the environment and placed in contact with the biosensor. If an agent is present in the sample, the molecular recognition elements are brought into sufficient proximity—called the Förster radius—to allow the transfer of energy from the donor to the recipient. For a detailed description of the principle of luminescence resonance energy transfer, see Thomas Heyduk, “Measuring protein conformational changes by FRET/LRET,” Current Opinion in Biotechnology, Vol. 13, Issue 4, Aug. 1, 2002, pp. 292-296, which is incorporated herein by reference. The biosensor—sample complex is placed into a hardware component containing a light source and photodetector. The light source produces light which excites—i.e. energizes—the donor. If the agent is present and the donor is in sufficient proximity to the recipient (i.e., r≦X, wherein X is the maximum distance between donor and recipient to allow for at least 10% efficiency of energy transfer between donor and recipient and r is the distance between the donor and recipient), the excited donor transfers its energy to the recipient, which becomes excited. The excited recipient then returns to its unexcited (ground) state and emits a light of a particular wavelength, which is detected by the photodetector. The Förster radius (R₀) defines the distance between a donor and acceptor at which energy transfer is 50% efficient. Each donor and acceptor pair has a particular R₀.

In a preferred embodiment, one or more of the molecular recognition elements is an antibody that recognizes and is capable of binding to a particular and specific position on the agent. More preferably, each molecular recognition element is an antibody, each of which recognizes and is capable of binding to a its particular and specific position on the agent, wherein each position is close enough to another position to allow the donor, which is bound to a first antibody, to be in close enough proximity to the recipient, which is bound to a second antibody, to allow for the transfer of energy from the donor to the recipient, and the concomitant emission of light of a particular wavelength from the recipient. Preferably, the agent is a microbe (e.g., bacteria, virus, fungus, protist) or a toxin, and the sample is a food sample.

In a preferred embodiment, the donor has a long-lived luminescence profile, i.e., greater than 10 microseconds, more preferably greater than 100 microseconds, and most preferably greater than one (1) millisecond. This attribute enables greater signal to noise ratio and better sensitivity by allowing the light source to be turned on to excite the donor, then turned off prior to collecting data from the photodetector. When the light source is turned off, the donor continues to transfer stored energy to the recipient, which in turn emits (fluoresces) light of a particular wavelength range, which is detected at the photodetector. Thus, any autofluorescence from the sample has an opportunity to decay before the recipient fluorescence is detected. This is called Time-Resolved Luminescence Resonance Energy Transfer (TR-LRET). More preferably, the donor is a lanthanide series metal and organic molecule complex, such as a terbium chelate (e.g., terbium in a complex with diethylenetriaminepentaacetic acid [DPTA]), a europium chelate, and the like. For a ore detailed discussion of TR-LRET, see Selvin and Hearst, “Luminescence energy transfer using a terbium chelate: improvements on fluorescence energy transfer,” Proc. Natl. Acad. Sci. USA, volume 91, pp. 1024-1028, October 1994, which is incorporated herein by reference. Most preferably, the donor is a terbium chelate and the recipient is an organic fluorophore, such as for example but not limited to a fluorecein, fluorescein derivative, rhodamine, rhodamine derivative, Cy3, or Cy5.

In another embodiment, each molecular recognition element is linked to a substrate, such as for example a silicon chip, a plastic surface, a glass slide, glass beads, plastic beads, or a polymer strand.

In another embodiment, a first element is joined to a second element by way of a flexible molecular linker, creating essentially a bivalent molecular recognition element, which enables the rapid and more highly avid association of the first and second element to the agent. This bivalent molecular element may be free in solution or bound to a substrate.

In yet another embodiment, the biosensor component comprises multiple different molecular recognition elements, each capable of binding to a different agent. Such a multiplexed biosensor component is capable of detecting multiple agents in a single step in a single assay. In one aspect, each agent-specific molecular recognition pair is located at a specific address on the substrate, such that the device interprets spatial as well as light intensity information, wherein each address represents a particular agent. In another aspect, each agent-specific molecular recognition element pair is labeled with a different LRET pair, which allows for each specific agent binding event to produce a specific wavelength of emitted light. The device differentiates each particular agent according to the intensity of light of a particular wavelength.

For example, a multiplex biosensor that is useful to the food biosafety applications may include molecular recognition pairs directed to Salmonella, E. coli, Campylobacter and Listeria monocytogenes, which enables the user to measure any and all of those pathogens in a single food sample in a single test.

### EXAMPLE

### TR-LRET Sensor for E. coli

A biosensor for E. coli was developed using monoclonal antibodies directed to obtained from QED Bioscience, Inc. (catalogue numbers 15402, 15403, 15404, and 15405.) The antibodies, four (4) in total, specifically recognize E. coli serotypes O2a/2b, O7, O11, O18, O44, O112, and O125, and were demonstrated not to cross react with Enterobacter aerogenes, Klebsiella pneumoniae, Proteus mirabilis, Proteus vulgaris, Pseudomonas aeruginosa, and Serratia marcescens. Antibodies 15402 and 15403 were pooled to a final concentration of 1 mg/ml each in physiological phosphate buffered saline to create molecular recognition element A (“MRE-A”). Likewise, antibodies 15404 and 15405 were pooled to create molecular recognition element B (“MRE-B”).

100 ug of MRE-A was conjugated with 10 ug of terbium chelate according in general to Li, M. and Selvin, P. R. (1997) Bioconjug. Chem. 8:127-132, and “LanthaScreen™ TR-FRET Labeling Reagents Protocol,” Invitrogen publication lit. #762-038205, Nov. 3, 2003, which are incorporated herein by reference. The terbium chelate was a carbostyril 124-diethyletriamine-pentaacetic acid (CS124-DTPA)-based terbium chelate (CS124-DTPA-Phe-NCS.Tb, aka “CDPN.Tb”) (PanVera/Invitrogen Cat. No. P3055). The MRE-A.CDPN.Tb conjugate was purified on a G-50 molecular sieve column.

100 ug of MRE-B was conjugated to Cy3™ monofunctional NHS-Ester (“Cy3”) (Amersham Biosciences Cat. No. PA33001) according to manufacturer's instructions PA33001PLREv-D, 2003, which is herein incorporated by reference. In summary, 5 ul of coupling buffer (1M sodium carbonate, pH9.3) was mixed with 100 ug of MRE-B in 100 ul phosphate buffer saline. This mixture was added to the reactive dye vial containing dried Cy3 and mixed gently but thoroughly. This mixture incubated at room temperature for 30 minutes to form the MRE-B.Cy3 conjugate. The MRE-B.Cy3 conjugate was purified on a G-50 molecular sieve column.

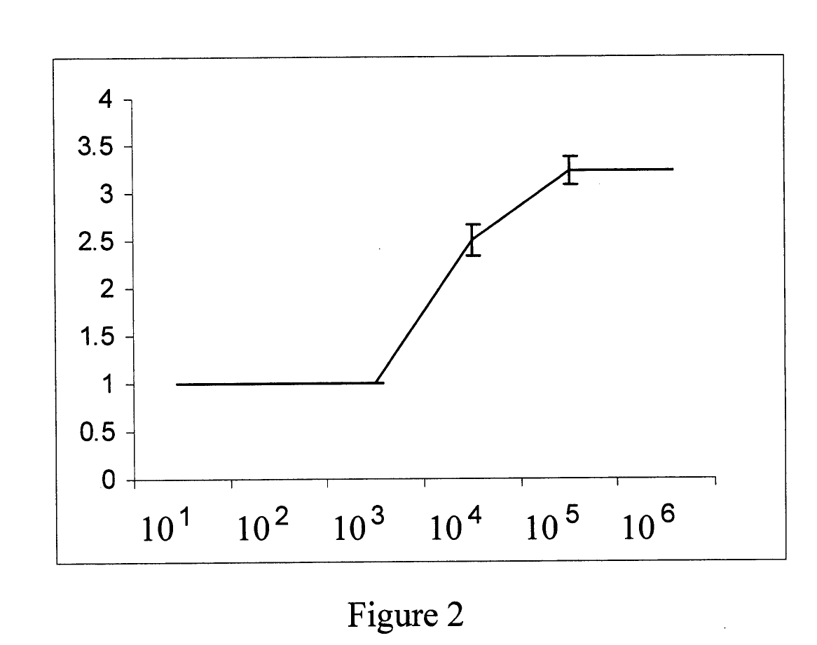

A ten-fold serial dilution of E. coli was made into 500 ul of phosphate buffered saline containing 0.5 ul each of MRE-A.CDPN.Tb conjugate and MRE-B.Cy3 conjugate. The serial dilutions ran from 10¹ bacteria to 10⁵ bacteria (FIG. 2, X-axis). Within between 5 to 15 minutes of mixing together the bacteria and conjugated MREs, each sample was subjected to fluorometric analysis on a nitrogen laser-based microsecond lifetime instrument (compliments of the Heyduk laboratory at Saint Louis University School of Medicine Department of Biochemistry and Molecular Biology). The samples were excited using a nitrogen laser (337 nm) at a 5 ns pulse width 30 times per second. Emission light at 541 nm, which corresponds to one of the emission wavelengths associated with terbium chelate, and 570 nm, which corresponds to the emission wavelength of Cy3, were detected after each pulse and summated. The ratio of 570/541 was calculated for each E. coli sample. Two independent experiments were performed. The results are depicted in FIG. 2, which shows a positive correlation between number of E. coli bacteria (X-axis) and adjusted 570/541 ratio (Y-axis). By adjusted, the baseline was set at 1.0 which is the mixture of the MRE-A conjugate and the MRE-B conjugate without any E. coli present.

Without wishing to be limited by theory, this experiment demonstrates that in the presence of increasing amounts of bacteria, the terbium (donor) signal is quenched as the Cy3 (recipient) signal increases, which provides proof-of-concept for the homogeneous detection of agents such as bacteria using antibody based TR-LRET.

What is claimed is:

1. A device comprising a first molecular recognition element labeled with a luminescence donor and a second molecular recognition element labeled with a luminescence acceptor, both recognition elements affixed to a substrate, wherein the first molecular recognition element can bind to an agent at a first spot on the agent and the second molecular recognition element can bind to the agent at a second spot on the agent such that, in the presence of the agent, the luminescence donor transfers energy to the luminescence acceptor and the luminescence acceptor emits a detectable signal.

2. The device according to claim 1 wherein the first and second molecular recognition elements are a first and second antibody, respectively, wherein the first antibody and the second antibody bind to different but proximal epitopes on the agent, wherein the epitopes are within a Förster radius of each other.

3. The device according to claim 1 wherein the donor is a lanthanide chelate and the acceptor is an organic dye.

4. The device according to claim 1 wherein the substrate is selected from the group consisting of flexible polymer, silicon chip, glass slide, and swab.

5. The device according to claim 1 wherein the substrate is a glass slide.

6. A biosensor comprising a first molecular recognition element labeled with a luminescence donor and a second molecular recognition element labeled with a luminescence acceptor, wherein the first molecular recognition element can bind to an agent at a first spot on the agent and the second molecular recognition element can bind to the agent at a second spot on the agent such that, in the presence of the agent, the luminescence donor transfers energy to the luminescence acceptor and the luminescence acceptor emits a detectable signal.

7. The biosensor according to claim 6 wherein the first molecular recognition element is selected from the group consisting of aptamer, receptor, antibody and antibody fragment, and the second molecular recognition element is selected from the group consisting of aptamer, receptor, antibody and antibody fragment.

8. The biosensor according to claim 6 wherein the first and second molecular recognition elements are a first and second antibody, respectively, wherein the first antibody and the second antibody bind to different but proximal epitopes on the agent, wherein the epitopes are within a Förster radius of each other.

9. The biosensor according to claim 6 wherein the donor is a lanthanide chelate and the acceptor is an organic dye.

10. The biosensor according to claim 9 wherein the lanthanide is terbium and the acceptor is Cy3.

11. A method of detecting an agent in a sample comprising the steps of (a) applying a sample to a biosensor, wherein the biosensor comprises a first molecular recognition element labeled with a luminescence donor and a second molecular recognition element labeled with a luminescence acceptor, wherein the first molecular recognition element can bind to an agent at a first spot on the agent and the second molecular recognition element can bind to the agent at a second spot on the agent such that, in the presence of the agent, the luminescence donor transfers energy to the luminescence acceptor and the luminescence acceptor emits a detectable signal, (b) exposing the biosensor to a pulse of light which excites the donor, (c) quantitatively detecting light emitted from the acceptor, and (d) estimating the amount of agent present in the sample.

12. The method according to claim 11 wherein the agent is a bacteria and the sample is a food sample.

13. The method according to claim 11 wherein the first and second molecular recognition elements are a first and second antibody, respectively, wherein the first antibody and the second antibody bind to different but proximal epitopes on the agent, wherein the epitopes are within a Förster radius of each other

14. The method according to claim 11 wherein the donor is a lanthanide chelate and the acceptor is an organic dye.

15. The method according to claim 11 wherein the pulse of light which excites the donor is ultraviolet and the light emitted from the acceptor is visible or infrared.

16. The method according claim 14 wherein the lanthanide is terbium and the acceptor is Cy3.
